(19) United States
(12) Patent Application Publication
(10) Pub. No.: US 2010/0034752 A1
(43) Pub. Date: Feb. 11, 2010

(54) INHALANT FORMULATIONS COMPRISING A BISPHOSPHONATE AND A PYRAZOLONE DERIVATIVE AND METHODS FOR USING THE SAME

(76) Inventors: Toru Hibi, San Jose, CA (US); Akira Yamamoto, San Jose, CA (US); Hidemasa Katsumi, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/330,749

(22) Filed: Dec. 9, 2008

Related U.S. Application Data
(60) Provisional application No. 61/087,956, filed on Aug. 11, 2008.

(51) Int. Cl. A61K 9/12; A61K 31/66

(52) U.S. Cl. 424/45; 514/103

(57) ABSTRACT

The present invention provides for methods of administering by a pulmonary route a bisphosphonate active agent in combination with a pyrazolone derivative to a subject. Also provided are pharmaceutical compositions for use in practicing methods according to embodiments of the invention. The methods and compositions according to embodiments of the invention find use in a variety of different applications, including but not limited to, the treatment of bone absorption disease conditions.

### CROSS-REFERENCE TO RELATED APPLICATIONS

Pursuant to 35 U.S.C. §119 (e), this application claims priority to the filing dates of: U.S. Provisional Patent Application Ser. No. 61/087,956 filed on Aug. 11, 2008; the disclosures of which applications are herein incorporated by reference.

### INTRODUCTION

Bisphosphonates and their pharmacologically acceptable salts find use in a variety of different applications. For example, bisphosphonates have been employed as bone absorption inhibitors in treating patients suffering from osteoporosis, Paget's disease and cancer.

Typically, bisphosphonates have been administrated orally and intravenously. However, there are disadvantages associated with the oral and intravenous administration of bisphosphonates. For example, the bioavailability of a bisphosphonate following oral administration can be very low. Because of the low bioavailability, high and/or more frequent oral doses may be required, which may cause problems with patient compliance to the treatment regimen. Furthermore, bisphosphonates can be irritating to the gastrointestinal tract. Consequently, this may cause further problems with patient compliance because patients are typically required to fast and remain upright following oral administration to avoid potential gastrointestinal side effects.

Intravenous administration of bisphosphonates, while overcoming some of the disadvantages of oral administration, is not entirely satisfactory. For example, because rapid intravenous administration of bisphosphonates may cause renal complications, intravenous bisphosphonates are generally administered slowly over many hours with careful monitoring of renal function.

Because of the above disadvantages of oral and intravenous bisphosphonate administration, inhalation administration of bisphosphonates has been proposed. See, e.g., U.S. Pat. No. 6,743,414. However, inhalation administration of bisphosphonates can be damaging to the pulmonary mucosal tissue.

### SUMMARY

The present invention provides for methods of administering by a pulmonary route a bisphosphonate active agent in combination with a pyrazolone derivative mucosal membrane protecting agent to a subject. Also provided are pharmaceutical compositions for use in practicing methods according to embodiments of the invention. The methods and compositions according to embodiments of the invention find use in a variety of different applications, including but not limited to, the treatment of bone absorption disease conditions.

### BRIEF DESCRIPTION OF THE FIGURES

FIG. 1 provides a graph of the observed LDH activity in bronchoalveolar lavage fluid (BALF) after intrapulmonary administration of phosphate buffer solution (PBS), alendronate, and alendronate in combination with edaravone in rats, as reported in the Experimental Section, below.

FIG. 2 provides a graph of plasma concentration-time profiles of alendronate (ALN) after its intrapulmonary administration in rats.

FIG. 3 provides pharmacokinetic parameters of alendronate (ALN) after its administration in rats.

### DEFINITIONS

When describing the compounds, pharmaceutical compositions containing such compounds, and methods of using such compounds and pharmaceutical compositions, the following terms have the following meanings unless otherwise indicated. It should also be understood that any of the moieties defined below may be substituted with a variety of substituents, and that the respective definitions are intended to include such substituted moieties within their scope.

“Alkyl” refers to monovalent saturated aliphatic hydrocarbyl groups particularly having up to 10 carbon atoms, or up to 9 carbon atoms, up to 8 carbon atoms, up to 6 carbon atoms, up to 3 carbon atoms, or one carbon atom. The hydrocarbon chain may be either straight-chained or branched. This term is exemplified by groups such as methyl, ethyl, n-propyl, isopropyl, n-butyl, isobutyl, sec-butyl, tert-butyl, n-hexyl, n-octyl, tert-octyl, and the like. The term “alkyl” also includes “cycloalkyl” as defined herein.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclohexyl, cyclooctyl, 1-methylcyclopropyl, 2-methylcyclopentyl, 2-methylcyclooctyl, and the like.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of such heterocyclic non-aromatic rings include, but are not limited to, aziridinyl, azetidinyl, piperazinyl, and piperidinyl.

“Aryl” refers to a monovalent aromatic hydrocarbon group derived by the removal of one hydrogen atom from a single carbon atom of a parent aromatic ring system. Typical aryl groups include, but are not limited to, groups derived from benzene, ethylbenzene, mesitylene, toluene, xylene, aniline, chlorobenzene, nitrobenzene, and the like.

“Aralkyl” or “arylalkyl” refers to an alkyl group, as defined above, substituted with one or more aryl groups, as defined above.

“Heteroaryl” refers to a stable heterocyclic aromatic ring and fused rings containing one or more heteroatoms independently selected from N, O and S. A fused heterocyclic ring system may include carbocyclic rings and need only include one heterocyclic ring. Examples of such heterocyclic aromatic rings include, but are not limited to, pyridine, pyrimidine, and pyrazinyl.

“Halogen” refers to fluoro, chloro, bromo and iodo. In some embodiments, the halogen is fluoro or chloro.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryl, substituted thioaryl, thioketo, thiol, alkyl-S(O)—, aryl-S(O)—, alkyl-S(O)₂— and aryl-S(O)₂—.

### DETAILED DESCRIPTION

The present invention provides for methods of administering by a pulmonary route a bisphosphonate active agent in combination with a pyrazolone derivative mucosal membrane protecting agent to a subject. Also provided are pharmaceutical compositions for use in practicing methods according to embodiments of the invention. The methods and compositions according to embodiments of the invention find use in a variety of different applications, including but not limited to, the treatment of bone absorption disease conditions.

Before the present invention is described in greater detail, it is to be understood that this invention is not limited to the particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

In further describing the subject invention, the subject methods are described first in greater detail, followed by a review of the various compositions, e.g., formulations and kits, that may find use in the subject methods, as well as a discussion of various representative applications in which the subject methods and compositions find use.

### Methods

Aspects of the invention include methods of administering a bisphosphonate active agent in combination with a pyrazolone derivative, which may be viewed as a mucosal membrane protecting agent, to a subject. The subject may be in need thereof, e.g., for the treatment of a disease or condition treatable by a bisphosphonate active agent (as described in greater detail below).

The term “in combination with” means that an amount of the pyrazolone derivative is administered anywhere from simultaneously to up to 5 hours or more, e.g. 10 hours, 15 hours, 20 hours or more, prior to or after the administration of the bisphosphonate active agent. In certain embodiments, the bisphosphonate active agent and pyrazolone derivative are administered sequentially to the subject, e.g., where the bisphosphonate active agent is administered before or after the pyrazolone derivative. In other embodiments, the bisphosphonate active agent and pyrazolone derivative are administered simultaneously to the subject, e.g., where the bisphosphonate active agent and pyrazolone derivative are administered to the subject at the same time as two separate formulations, or are combined into a single formulation that is administered to the subject. Regardless of whether the bisphosphonate active agent and pyrazolone derivative are administered sequentially or simultaneously, as illustrated above, the agents are considered to be administered together or in combination (i.e., in conjunction) for purposes of the present invention. Routes of administration of the two agents may vary, where routes of administration of interest include, but are not limited to, those described in greater detail below.

### Bisphosphonate Active Agent

In the subject methods, a bisphosphonate active agent is administered to a subject in combination with a pyrazolone. Bisphosphonate active agents of interest include bisphosphonate compounds that are capable of inhibiting the resorption of bone. Bisphosphonate compounds are also known as diphosphonates or bisphosphonic acids. The bisphosphonate active agent may have a high affinity for bone tissue. In some embodiments, the bisphosphonate active agent metabolizes in a cell into compounds that compete with adenosine triphosphate (ATP) in the cellular energy metabolism pathway. In some embodiments, the bisphosphonate active agent binds the farynesyl disphosphate synthase (FPPS) enzyme and inhibits the enzymatic activity of FPPS. FPPS is an enzyme involved in the 3-hydroxy-3-methylglutaryl-CoA (HMG-CoA) reductase pathway (or mevalonate pathway). Bisphosphonate active agents useful in the subject compositions include, but are not limited to those compounds described in U.S. Pat. Nos. 4,621,077; 5,183,815; 5,358,941; 5,462,932; 5,661,174; 5,681,590; 5,994,329; 6,015,801; 6,090,410; 6,225,294; 6,414,006; 6,482,411; and 6,743,414; the disclosures of which are herein incorporated by reference.

In certain embodiments, the bisphosphonate active agent is a compound of formula (I):

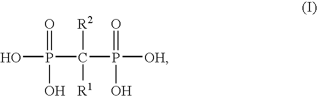

or a physiologically acceptable salt, solvate, hydrate, and prodrug forms thereof, and stereoisomers thereof;

wherein:

R¹ is a hydrogen, —OH, or a halogen; and

R² is a halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein each carbon atom of R² may be optionally replaced with a nitrogen or sulfur atom and R² has no more than 3 nitrogen or sulfur atoms in total.

In certain embodiments, R² is a halogen, a linear or branched substituted or unsubstituted C₁-C₉ alkyl, a linear or branched substituted or unsubstituted C₁-C₉ cycloalkyl, a linear or branched substituted or unsubstituted C₁-C₉ aryl, or a linear or branched substituted or unsubstituted C₁-C₉ aralkyl, wherein the each carbon atom of R² may be optionally replaced with a nitrogen or sulfur atom and R² has no more than 2 nitrogen or sulfur atoms in total, wherein R² has no more than 8 carbon atoms.

In certain embodiments, R² is a linear or branched C₁-C₈ alkyl, wherein the each carbon atom of R² may be optionally replaced with a nitrogen atom and the total number of nitrogens in R² is not more than 1, wherein the C₁-C₈ alkyl may be optionally substituted with an amino group.

In some embodiments, R¹ is a hydroxy or a fluorine and R² is a fluorine or a linear or branched C₁-C₅ alkyl, which may be optionally substituted with substituents such as amino groups and/or fluorine atoms, and their salts with alkali metals, organic bases and basic amino acids. In some embodiments, R² is:

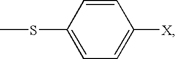

wherein X is a halogen.

In some embodiments, R² is:

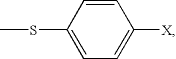

wherein X is a halogen; and R¹ is hydrogen.

In some embodiments, R² is —CH₃, —CH₂—CH₂—NH₂, —(CH₂)₃—NH₂, —(CH₂)₅—NH₂, —(CH₂)₂—N(CH₃)₂,

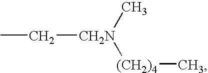

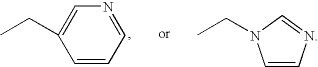

Specific bisphosphonate active agents of interest are shown in Table 1 (wherein the compound is of formula (I)):

TABLE 1

| Bisphosphonate | | |
|---|---|---|
| active agent | R¹ side chain | R² side chain |
| etidronate | —OH | —CH₃ |
| clodronate | —Cl | —Cl |
| tiludronate | —H | 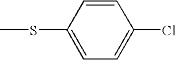 |
| pamidronate | —OH | —CH₂—CH₂—NH₂ |
| neridronate | —OH | —(CH₂)₅—NH₂ |
| olpadronate | —OH | —(CH₂)₂—N(CH₃)₂ |
| alendronate | —OH | —(CH₂)₃—NH₂ |
| ibandronate | —OH | 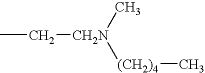 |
| risedronate | —OH | 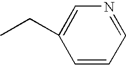 |
| zoledronate | —OH | 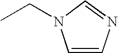 |

In certain embodiments, the bisphosphonate active agent of interest is 4-amino-1-hydroxybutane-1,1-bisphosphonic acid (alendronate; CAS Registry No. 121268-17-5), represented by formula (II), as follows:

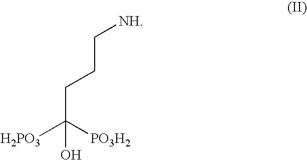

Additional specific bisphosphonates of interest include, but are not limited to: (dichloromethylene)bisphosphonic acid (clodronate; CAS Registry No. 10596-23-3); (1-hydroxyethylidene)bisphosphonic acid (etidronate; CAS Registry No. 7414-83-7); (1-hydroxy-3-(methylpentylamino)propylidene)bisphosphonic acid (ibandronate; CAS Registry No. 114084-78-5); ((cycloheptylamino)methylene)bisphosphonic acid (incadronate; CAS Registry No. 124351-85-5); (1-hydroxy-2-(imidazo(1,2-a)-pyridin-3-yl)ethylidene)bisphosphonic acid (minodronate; CAS Registry No. 127657-42-5); (6-amino-1-hydroxyhexylidene)bisphosphonic acid (neridronate; CAS Registry No. 79778-41-9); (3-(dimethylamino)-1-hydroxypropylidene)bisphosphonic acid (olpadronate; CAS Registry No. 63132-39-8); (3-amino-1-hydroxypropylidene)bisphosphonic acid (pamidronate; CAS Registry No. 57248-88-1); (1-hydroxy-2-(3-pyridinyl)ethylidine)bisphosphonic acid (risedronate; CAS Registry No. 105462-24-6); (((4-chlorophenyl)thio)methylene)bisphosphonic acid (tiludronate; CAS Registry No. 89987-06-4); (1-hydroxy-2-(1 H-imidazole-1-yl)ethylidene)bisphosphonic acid (zoledronate; CAS Registry No. 118072-93-8); ((cycloheptylamino)methylene)bisphosphonic acid (incadronate; CAS Registry No. 124351-85-5); 5-amino-1-hydroxypentane-1,1-bisphosphonic acid; difluoro-methanbiphosphonic acid (CAS Registry No. 10596-32-4); and pharmacologically acceptable salts thereof.

Pharmacologically acceptable salts include, but are not limited to, salts of alkali metals (e.g., sodium and potassium), salts of alkali earth metals (e.g., calcium), salts of inorganic acids (e.g., HCl), and salts of organic acids (e.g., citric acids and amino acids, such as lysine). In one embodiment, the bisphosphonate active agent is a salt of sodium. When the bisphosphonate active agent is alendronate, the monosodium salt trihydrate form of alendronate is employed in certain embodiments. In certain embodiments, the bisphosphonate active agent is in its anhydrous form.

### Pyrazolone Derivative

As summarized above, the bisphosphonate active is administered in combination with a pyrazolone derivative. In certain embodiments, the pyrazolone derivative may be viewed as a mucosal membrane protecting agent. The term “mucosal membrane protecting agent” refers to an agent that reduces unwanted irritation caused by the bisphosphonate active agent when the bisphosphonate active agent is administered to a subject by a pulmonary route. As such, a mucosal membrane protecting agent is one that reduces bisphosphonate induced pulmonary irritation. Mucosal membrane protecting agents of interest are those agents that reduce bisphosphonate induced pulmonary irritation by about 2 to 10-fold or more, such as by about 50-fold or more, and including by about 100-fold or more, as determined using, for example an in situ trans-pulmonary absorption test and/or a pulmonary inflammation test, as compared to a suitable control, e.g., bisphosphonate by itself as the sole active agent in an inert delivery vehicle.

In certain embodiments, the pyrazolone derivative is a compound of formula (III):

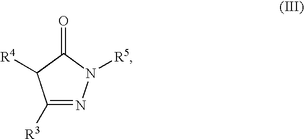

or a physiologically acceptable salt, solvate, hydrate, and prodrug forms thereof, and stereoisomers thereof;

wherein:

R³ is a hydrogen, an aryl, an alkyl having 1 to 5 carbon atoms, or an alkoxycarbonylalkyl having 1 to 6 carbon atoms in total;

R⁴ is a hydrogen, an aryloxy, an arylmercapto, an alkyl having 1 to 5 carbon atoms, or a hydroxyalkyl having 1 to 3 carbon atoms; or R³ and R⁴ are coupled together to form an alkylene having 3 to 5 carbon atoms; and

R⁵ is a hydrogen, an alkyl having 1 to 5 carbon atoms, a cycloalkyl having 5 to 7 carbon atoms, a hydroxyalkyl having 1 to 3 carbon atoms, a benzyl, a naphthyl, or a substituted or unsubstituted phenyl.

In some embodiments, R³ is an alkyl having 1 to 5 carbon atoms; R⁴ is a hydrogen; and R⁵ is an unsubstituted phenyl, or a phenyl substituted by 1 to 3 substituents, which may be the same or different and selected from the group consisting of an alkyl having 1 to 6 carbon atoms, an alkoxy having 1 to 5 carbon atoms, a hydroxyalkyl having 1 to 3 carbon atoms, an alkoxycarbonyl having 2 to 5 carbon atoms in total, an alkylmercapto having 1 to 3 carbon atoms, an alkylamino having 1 to 4 carbon atoms, a dialkylamino having 2 to 8 carbon atoms in total, a halogen atom, trifluoromethyl, carboxyl, cyano, hydroxyl group, nitro, amino, sulfonyl, and acetamido.

In some cases, R³ is an alkyl having 1 to 5 carbon atoms; R⁴ is a hydrogen; and R5 is an unsubstituted phenyl.

In certain embodiments the pyrazolone derivative is 3-methyl-1-phenyl-pyrazolin-5-one (edaravone; trade name “Radicut” (manufactured and sold by Mitsubishi Pharma Corporation); CAS Registry No. 89-25-8), represented by formula (IV), as follows:

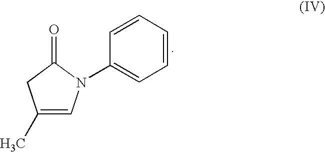

Additional specific pyrazolone derivative mucosal membrane protecting agents of interest include, but are not limited to: 3-methyl-1-(4-methylphenyl)-pyrazolin-5-one (CAS Registry No. 86-92-0); 3-(ethoxycarbonyl)-1-phenyl-pyrazolin-5-one (CAS Registry No. 89-33-8); 3-methyl-1-(4-sulfophenyl)-2-pyrazolin-5-one (CAS Registry No. 89-36-1); 1-(3-chlorophenyl)-3-methyl-2-pyrazolin-5-one (CAS Registry No. 90-31-3); 1-(2-chlorophenyl)-3-methyl-2-pyrazolin-5-one (CAS Registry No. 14580-22-4); 1-phenyl-3-carboxy-pyrazolin-5-one (CAS Registry No. 119-18-6); 1-(4-((2-hydroxyethyl)sulfonyl)phenyl)-3-methyl-2-pyrazolin-5-one (CAS Registry No. 21951-34-8); and pharmacologically acceptable salts thereof.

In certain embodiments, one or more additional mucosal membrane protecting agents may also be administered to a subject, such as protecting enzymes and protecting peptides as described in U.S. patent application Ser. No. 11/935,764, the disclosure of which is herein incorporated by reference.

As indicated above, an effective amount of the pyrazolone derivative mucosal membrane protecting agent(s) is employed in the subject methods. In certain embodiments, the amount of pyrazolone derivative employed is not more than about the amount of the bisphosphonate active agent employed. In other embodiments, the effective amount is the same as the amount of the active agent, and in certain embodiments the effective amount is an amount that is more than the amount of the bisphosphonate active agent. Effective amounts can readily be determined empirically.

In some embodiments, the bisphosphonate active agent may be alendronate, and the mucosal membrane protecting agent may be a chemical protecting agent, such as a pyrazolone derivative mucosal membrane protecting agent. In these embodiments, the pyrazolone derivative mucosal membrane protecting agent may be edaravone, such that alendronate is administered in combination with edaravone.

### Formulations and Administration

Also provided are pharmaceutical compositions containing the bisphosphonate active agent and/or pyrazolone derivative employed in the subject methods. In certain embodiments, the bisphosphonate active agent and/or pyrazolone derivative, e.g., in the form of a pharmaceutically acceptable salt, are formulated for pulmonary administration to a subject. In certain embodiments, e.g., where the compounds are administered as separate formulations (such as in those embodiments where they are administered sequentially), separate or distinct pharmaceutical compositions, each containing a different active agent, are provided. In some embodiments, a single formulation that includes both the bisphosphonate active agent and the pyrazolone derivative (i.e., one composition that includes both active agents) is provided.

By way of illustration, the bisphosphonate active agent and/or pyrazolone derivative can be admixed with conventional pharmaceutically acceptable carriers and excipients (i.e., vehicles) and used in forms suitable for pulmonary administration. Such suitable forms include aqueous solutions, suspensions, and the like. Such pharmaceutical compositions contain, in certain embodiments, from about 0.1% to about 90% by weight of the active compound(s), such as from about 1% to about 60%, including from about 1% to about 30% by weight of the active compound(s).

A liquid composition may be present as a suspension or a solution of the compound or pharmaceutically acceptable salt in a suitable liquid carrier(s), such as but not limited to glycerine, sorbitol, non-aqueous solvents such as but not limited to polyethylene glycol, oils, or water, with a suspending agent, preservative, surfactant, wetting agent, flavoring, or coloring agent, or the like. Alternatively, a liquid formulation can be prepared from a reconstitutable powder. The terms “reconstitutable” and “reconstitute” mean to return a substantially dry or dehydrated compound or mixture of compounds to a liquid state by adding a suitable solvent or water.

In certain embodiments of interest, the bisphosphonate active agent and the pyrazolone derivative are administered as a single pharmaceutical formulation, that, in addition to including an effective amount of each of the agents, includes other suitable compounds and carriers, and also may be used in combination with other active agents. The present invention, therefore, also includes pharmaceutical compositions comprising pharmaceutically acceptable excipients. Pharmaceutically acceptable excipients may include, for example, any suitable vehicles, adjuvants, carriers or diluents, and are readily available to the public. The pharmaceutical compositions of the present invention may further contain other active agents as are well known in the art.

One skilled in the art will appreciate that a variety of suitable methods of administering a formulation of the present invention to a subject are available, and although more than one route can be used to administer a particular formulation, a particular route can provide a more immediate and more effective reaction than another route. Pharmaceutically acceptable excipients may be employed as desired. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting.

In certain embodiments, the subject formulations of the present invention can be made into aerosol formulations to be administered via inhalation. In some cases, the pharmaceutical composition is an aerosol of liquid particles. In other cases, the pharmaceutical composition is an aerosol of solid particles. In these cases, the aerosol of solid particles may be a dry powder. The subject aerosol formulations (i.e., inhalant formulations) of the present invention can be formulated for use with acceptable pressurized propellants, such as dichlorodifluoromethane, propane, nitrogen, and the like. The subject aerosol formulations may also be formulated as pharmaceuticals for non-pressured preparations, such as for use in a nebulizer or an atomizer.

Those of skill in the art will readily appreciate that dose levels can vary as a function of the specific compound, the nature of the delivery vehicle, and the like. Suitable dosages for a given compound are readily determinable by those of skill in the art by a variety of means.

The dose administered to an animal, particularly a human, in the context of the present invention should be sufficient to facilitate a prophylactic or therapeutic response in the animal over a reasonable time frame. One skilled in the art will recognize that dosage will depend on a variety of factors including the strength of the particular compound employed, the bioavailability of the compound, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to bone absorption inhibiting agents that are known to reduce bone loss due to bone absorption, particularly unmodified bisphosphonate. A suitable dosage is an amount which results in the inhibition of bone absorption, without significant side effects. In proper doses and with suitable administration of certain compounds, the present invention provides for a wide range of intracellular effects, e.g., from partial inhibition to essentially complete inhibition of bone absorption.

Optionally, the pharmaceutical composition may contain other pharmaceutically acceptable components, such a buffers, surfactants, viscosity modifying agents, preservatives and the like. Each of these components is well-known in the art. See, e.g., U.S. Pat. No. 5,985,310, the disclosure of which is herein incorporated by reference. Other components suitable for use in the formulations of the present invention can be found in Remington's Pharmaceutical Sciences, Mace Publishing Company, Philadelphia, Pa., 17th ed. (1985).

In certain embodiments, the formulations of the present invention are administered to the host by a pulmonary route. In some embodiments, the pulmonary route of administration is in an inhalation dosage form directly into the respiratory tract, or directly to the respiratory airway, trachea, bronchi, bronchioles, lungs, alveolar ducts, alveolar sacs, and/or alveoli. The formulations may be administered by any convenient method, such as but not limited to: metered dose inhalers, nebulizers, atomizers, breath-activated inhalers or dry powder inhalers. The methods of the present invention also include administrating the formulations directly into the nasal cavity or oral cavity of the host with a dropper, pipette or cannula.

In certain embodiments, the formulation is in a powder form. For pharmaceutical purposes the average particle size of the powder may be no greater than about 100 μm in diameter. In certain embodiments, the average particle size of the finely-divided solid powder is about 25 μm or less, such as about 10 μm or less in diameter. The agents may be used as a powder with an average particle size ranging from about 1 μm to about 10 μm, such as from about 2 μm to about 8 μm, including about 2 μm to about 6 μm. The average particle size of the powder for inhalation therapy may range from about 1 μm to about 10 μm.

The concentration of active agent depends upon the desired dosage. The precise therapeutic dosage amount depends on the age, size, sex and condition of the subject, the nature and severity of the disorder, and other such factors. An ordinarily skilled physician or clinician can readily determine and prescribe the effective amount of the drug required for a particular patient.

In some embodiments, the formulations are powdered aerosol formulations which include the active agents suspended or dispersed in a propellant or a propellant and solvent. The propellant generally comprises a mixture of liquefied chlorofluorocarbons (CFCs) which are selected to provide the desired vapor pressure and stability of the formulation. Widely used propellants in aerosol formulations for inhalation administration include, but are not limited to Propellant 11 (trichlorofluoromethane; CAS Registry No. 91315-61-6), Propellant 12 (dichlorodifluoromethane; CAS Registry No. 75-71-8), Propellant 114 (1,2-dichloro-1,1,2,2-tetrafluoroethane; CAS Registry No. 76-14-2). Other commonly used propellants include, but are not limited to Propellant 113 (1,1,2-trichloro-1,2,2-trifluoroethane; CAS Registry No. 76-13-1), Propellant 142b (1-chloro-1,1-difluoroethane; CAS Registry No. 75-68-3), Propellant 152a (1,1-difluoroethane; CAS Registry No. 75-37-6), Propellant 124 (2-chloro-1,1,1,2-tetrafluoroethane; CAS Registry No. 2837-89-0), HFA-227ea (1,1,1,2,3,3,3-heptafluoropropane; CAS Registry No. 431-89-0), HFA-236fa (1,1,1,3,3,3 hexafluoropropane; CAS Registry No. 690-39-1), carbon dioxide propellant (CAS Registry No. 124-38-9), and dimethyl ether (CAS Registry No. 115-10-6), which are commercially available from DuPont FluroChemicals (Wilmington, Del.). The propellant HFA-134a (1,1,1,2-tetrafluoroethane; CAS Registry No. 811-97-2) is also a commonly used propellant for medicinal aerosol formulations. In certain embodiments, the propellant may comprise about 40% to 90% by weight of the total inhalation composition, such as about 50% to 80%, including about 60% to 70%.

The inhalation composition may also contain dispersing agents and solvents, such as phosphate buffer solution (PBS). Surfactants have also been used as dispersing agents. The surface active agents may be present in the weight ratio 1:100 to 10:1 surface active agent to bisphosphonate active agent, but the surface active agent may exceed this weight ratio in cases where the drug concentration in the formulation is very low. In some embodiments, the surface active agents may be present in amounts not exceeding 5% by weight of the total formulation.

The inhalation formulation of the present invention can be delivered in any convenient inhalation device, such as but not limited to an inhaler, a nebulizer or an atomizer.

In the methods and compositions of the present invention, the pharmaceutical composition may be administered in admixture with suitable pharmaceutical diluents, excipients or carriers. Moreover, when desired or necessary, suitable excipients, lubricants, disintegrating agents and coloring agents can also be incorporated into the mixture of active ingredient(s) and inert carrier materials.

In some embodiments, the pharmaceutical composition is a powder formulation comprising a bisphosphonate active agent, or pharmacologically acceptable salt thereof, and one or more mucosal membrane protecting agents. In some cases, the pharmaceutical composition includes an effective amount of both a bisphosphonate active agent (e.g., alendronate) and a pyrazolone derivative mucosal membrane protecting agent (e.g., edaravone) in a physiologically acceptable vehicle. In certain embodiments, the pharmaceutical composition further comprises one or more excipients, such as a plasticizer, lubricant, binder, disintegrator, stabilizer, or masking agent. In certain embodiments, the surface of the particles of the powder formulation are coated with a suitable coating agent. In certain cases, the pharmaceutical composition further comprises a lubricant, such as sucrose fatty acid ester or other substances which provide slippage between particles of the compound as well as lubrication for component parts of the valve of the inhalation device.

In some embodiments, the pharmaceutical composition is a solution or suspension formulation including a bisphosphonate active agent, or pharmacologically acceptable salt thereof, and one or more mucosal membrane protecting agents. In certain embodiments, the solution or suspension formulation includes the agents dissolved or suspended in water. In certain embodiments, the solution or suspension formulation further includes one or more co-solvents, such as but not limited to, ethanol, propylene glycol, and polyethylene glycol. In some cases, the solution or suspension formulation further comprises one or more preservatives, solubilizers, buffering agents, isotonizers, surfactants, absorption enhancers, or viscosity enhancers. In certain embodiments, the pharmaceutical composition is a suspension formulation and further comprises a suspending agent.

### Utility

The subject methods find use in a variety of applications, where in certain applications the methods are methods of modulating at least one cellular function, such as inhibiting bone reabsorption. The subject methods find use in treating, reducing the probability of, or preventing bone absorption, loss of bone mass, osteoporosis, osteopenia, urolithiasis, hypercalcemia, Paget's disease (or osteitis deformans), bone metastasis, multiple myeloma, neoplastic bone lesions, and other conditions that cause or increase the risk of bone fragility. In some embodiments of the invention, the subject methods are also useful for reducing the probability or risk of non-vertebral fractures. In certain embodiments, the subject in need of the bisphosphonate active agent is osteoroporotic or postmenopausal, or both. In certain embodiments, the subject is a woman who is osteoroporotic or postmenopausal, or both. In certain embodiments, the subject is a human juvenile with osteogenesis imperfecta.

In this respect, the subject methods and compositions find use in known applications of bisphosphonate, such as in treating diseases or disorders that are capable of being treated using bisphosphonate. Use of the subject compositions of the present invention is of particular utility in, for example, the treatment of diseases and disorders including but not limited to osteoporosis, osteopenia, urolithiasis, hypercalcemia, Paget's disease (or osteitis deformans), bone metastasis, multiple myeloma, neoplastic bone lesions, and other conditions that cause or increase the risk of bone fragility. In these capacities, use of the present inventive compositions will result in a reduced unwanted toxicity while retaining desired bisphosphonate activity.

As such, the subject methods and compositions find use in therapeutic applications in which bisphosphonate administration is indicated. A representative therapeutic application is the treatment of bone disease conditions, e.g., osteoporosis and related conditions characterized by bone absorption and loss of bone mass.

By “treatment” is meant that at least an amelioration of the symptoms associated with the condition afflicting the subject is achieved, where the term “amelioration” is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the subject no longer suffers from the condition, or at least the symptoms that are associated with the condition.

A variety of subjects are treatable according to the present methods. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be humans. In some embodiments, the subjects are women. In other embodiments, the subjects are men.

The methods disclosed herein find use in, among other applications, the treatment of bone disease conditions, including osteoporosis conditions. In such applications, an effective amount of the bisphosphonate active agent and pyrazolone derivative mucosal membrane protecting agent is administered to the subject in need thereof. Treatment is used broadly as defined above, e.g., to include at least an amelioration in one or more of the symptoms of the disease, as well as a complete cessation thereof, including a reversal and/or complete removal of the disease condition, e.g., cure.

Individuals may be diagnosed as being in need of the subject methods using any convenient protocol, and are generally known to be in need of the subject methods, e.g., they are suffering from a target disease condition or have been determined to be at risk for suffering from a target disease condition, prior to practicing the subject methods.

Particular applications in which the subject methods and compositions find use include those described in U.S. Pat. Nos. 4,621,077; 5,183,815; 5,358,941; 5,462,932; 5,661,174; 5,681,590; 5,994,329; 6,015,801; 6,090,410; 6,225,294; 6,414,006; 6,482,411; and 6,743,414; the disclosures of which are herein incorporated by reference.

### Kits & Systems

Also provided are kits that find use in practicing the subject methods, as described above. For example, kits and systems for practicing the subject methods may include one or more pharmaceutical formulations, which include one or both of the bisphosphonate active agent and pyrazolone derivative. As such, in certain embodiments the kits may include a single pharmaceutical composition, present as one or more unit dosages, where the composition includes both the bisphosphonate active agent and pyrazolone derivative. In yet other embodiments, the kits may include two or more separate pharmaceutical compositions, each containing one or more unit dosages of either a bisphosphonate active agent or a pyrazolone derivative mucosal membrane protecting agent.

The term “unit dosage”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the subject.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, computer-readable memory, etc., on which the information has been recorded or stored. Yet another means that may be present is a website address which may be used via the Internet to access the information at a removed site. Any convenient means may be present in the kits.

The term “system” as employed herein refers to a collection of bisphosphonate active agent(s) and mucosal membrane protecting agent(s) present in a single or disparate composition, that are brought together for the purpose of practicing the subject methods. For example, separately obtained bisphosphonate active agent(s) and mucosal membrane protecting agent(s) dosage forms brought together and coadministered to a subject, according to the present invention, are a system according to the present invention.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Experimental

### I. Pulmonary Inflammation Test

### A. Dosing Solution

12.5 mg/ml of alendronate (by Toronto Research Chemicals Inc.) for transpulmonary administration were prepared by using the isotonic phosphate buffer solution (PBS) with the pH of 7.4.

B. Dosing Solution Added with Mucosal Protective Agent

12.5 mg/ml of alendronate (by Toronto Research Chemicals Inc.) and 2 mg/ml of edaravone (by Toronto Research Chemicals Inc.) for transpulmonary administration were prepared by using the isotonic phosphate buffer solution (PBS) with the pH of 7.4.

### C. Pulmonary Inflammation Test

This test measures the degree of irritation caused by a drug to a subject's pulmonary tract following administration of the drug by the pulmonary route. Lactate dehydrogenase (LDH) activity was assayed using the LDH-Cytotoxic Test (Wako Pure Chemical Industries, Ltd., Osaka, Japan). LDH is a stable enzyme which is present in all cell types. When the plasma membrane of a cell is damaged, LDH is rapidly released from the cell. Measuring the level of LDH activity in the serum is the most widely used marker in cytotoxicity studies. A high level of LDH activity detected indicates a high degree of irritation, while a low level of LDH activity detected indicates a low degree of irritation.

Either phosphate buffered saline (PBS), alendronate (5 mg/kg), or alendronate (5 mg/kg) in combination with edaravone (0.8 mg/kg) was administered as a liquid formulation to a subject rat by the pulmonary route. Following administration of the liquid formulation, blood was removed from the aorta of the rat, and saline was injected from the pulmonary artery to wash the rat's lung with perfusion. The center of the neck was cut open to expose the bronchial tract, and a polyethylene tube was inserted to the bronchial tract to wash the bronchial tract with 16 mL of PBS (4 washes of 4 mL each) (bronchialveolar lavage (BAL)). The derived BAL fluid (BALF) was centrifuged at 4° C., 200×g for 7 minutes, and the supernatant was sampled to measure the LDH activity.

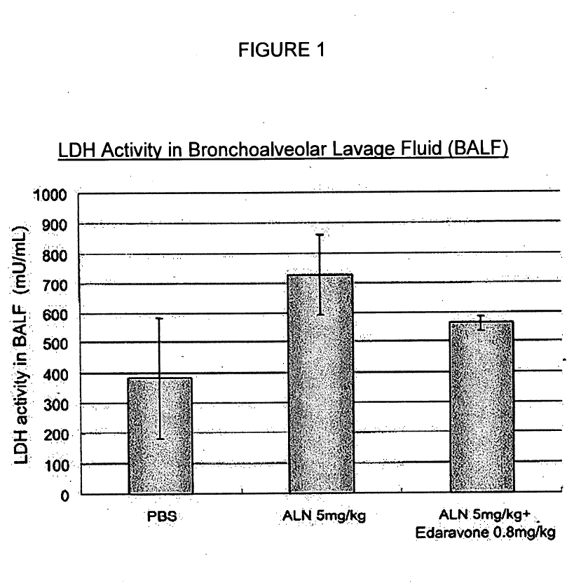

The results of this assay are provided in FIG. 1.

### II. Administration Route Analysis

### A. Dosing Solution

1.25 mg/ml of alendronate (by Toronto Research Chemicals Inc.) for venous administration was prepared by using an isotonic phosphate buffer solution (PBS) with a pH of 7.4.

12.5 mg/ml of alendronate (by Toronto Research Chemicals Inc.) and 12.5 mg/ml of alendronate +2 mg/ml edaravone (by Toronto Research Chemicals Inc.) for transpulmonary administration were prepared by using the isotonic phosphate buffer solution (PBS) with the pH of 7.4.

B. Transpulmonary Administration A transpulmonary absorption test was conducted as reported below (the following method is based on the method disclosed by Enna S J, Schanker L S. Absorption of saccharides and urea from the rat lung. Am. J. Physiol., 222, 409-414 (1972)).

A Wistar male rat weighing 250 to 300 g was used in the test. Under pentobarbital anesthesia, the center of the neck of the rat was cut open to expose the bronchial tract. A 2.5 cm long polyethylene tube (ID 1.5 mm, OD 2.3cm) was inserted from the thyroid cartilage between the 4^{th} and 5^{th} bronchial cartilage rings to a 0.6 cm depth, and the open skin was then stitched up. A 100 μl microsyringe (Microliter, no. 710, Hamilton Co) was filled with 100 μl of the dosing solution. The rat was placed at 80°. The tip of the microsyringe was inserted at 1 to 2 mm up into the bronchial tract through the above polyethylene tube and the solution was administered in sync with the breath of the rat in 1 to 2 seconds. Test formulations were administered to the rat by a pulmonary route. 45 seconds after the administration, the rat was placed at 10° and 250 μl of blood was sampled from the jugular vein in a time-dependent manner. The blood sample was centrifuged (13000 rpm, 10 min) to obtain the plasma fraction and it was stored at −30° right before the analysis.

### C. Venous Administration

A Wistar male rat weighing 250 to 300 g was used in the test. 1 mg/kg of Alendronate was administered to the rat through the femur vein. The blood sample was centrifuged (13000 rpm, 10 min) to obtain the plasma fraction and it was stored at −30° right before the analysis.

### D. Analysis Conditions

The assay was conducted in the following method in reference with the report by Wong et al., “Determination of Pamidronate in human whole blood and urine by reversed-phase HPLC with fluorescence detection,” Biomed. Chromatogy. (2004) 18: 98-101. 120 μl of the plasma fraction obtained from the rat was diluted with 500 μl of ultrapure water. 75 μl of trichloroacetic acid (TCA) was added to remove protein and the mixture was centrifuged (13000 rpm, 5 min). The supernatant was filtered with a filter (0.45 μm).

Calcium chloride and monobasic sodium phosphate were added to 600 μl of the filtered supernatant. Sodium hydroxide was added to adjust the pH to 12 to sediment. The mixture was centrifuged and the sediment was washed with 500 μl of ultrapure water. Hydrochloric acid was added to the sediment to dissolve and sodium hydroxide was added to obtain the precipitate. After centrifuging, it was washed with 500 μl of ultrapure water and the sediment was dissolved in 100 μl of 50 mM Na₂EDTA (pH 10). After adding 30 μl of a fluorescamine/acetonitrile solution (3 mg florescamine/ml acetonitrile), 100 μl of dichloromethane was added to stir vigorously and centrifuged (13,000 rpm, 5 min). The obtained supernatant was collected and 10 μl of it, as an injection volume, was measured with the fluorescent-reverse-phase HPLC under the following conditions.
Equipment Used: Shimadzu LC-10A system
Column: COSMOSIL C18 (4.6×150 mm)
Mobile Phase: 95% 1 mM Na₂EDTA-methanol ((97:3) pH 6.5 by 1N NaOH), 5% methanol
Flow Speed: 1.0 ml/min
Detector: Fluorescence detector (Ex: 395 nm, Em: 480 nm)
Column Temp.: 40°

### F. Results

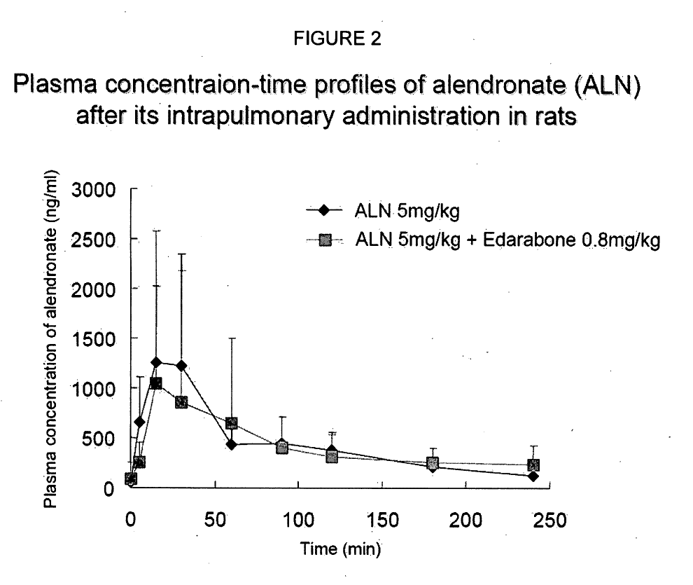

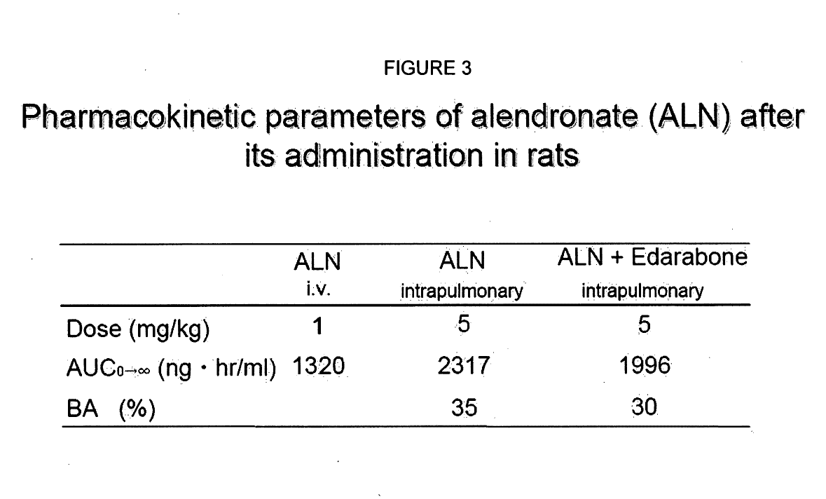

Results from the above analysis are shown in FIGS. 2 and 3. The results demonstrate that a combination of bisphosphonate and edaravone shows equivalent blood concentration of bisphosphonate to bisphosphonate alone.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of administering to a subject in need thereof an effective amount of a bisphosphonate active agent, said method comprising:
administering by a pulmonary route to said subject an effective amount of a bisphosphonate active agent in combination with a pyrazolone derivative.

2. The method according to claim 1, wherein said bisphosphonate active agent is a compound of formula (I): 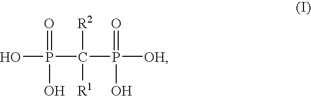
or a physiologically acceptable salt, solvate, hydrate, and prodrug forms thereof, and stereoisomers thereof;
wherein:
R¹ is a hydrogen, —OH, or halogen; and
R² is a halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein each carbon atom of R² may be optionally replaced with a nitrogen or sulfur atom and R² has no more than 3 nitrogen or sulfur atoms in total.

3. The method according to claim 2, wherein said bisphosphonate active agent is a compound listed in Table 1.

4. The method according to claim 3, wherein said bisphosphonate active agent is alendronate.

5. The method according to claim 1, wherein said pyrazolone derivative is a compound of formula (III): 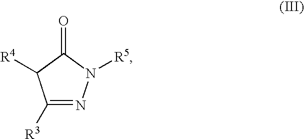
or a physiologically acceptable salt, solvate, hydrate, and prodrug forms thereof, and stereoisomers thereof;
wherein:
R³ is a hydrogen, an aryl, an alkyl having 1 to 5 carbon atoms, or an alkoxycarbonylalkyl having 1 to 6 carbon atoms in total;
R⁴ is a hydrogen, an aryloxy, an arylmercapto, an alkyl having 1 to 5 carbon atoms, or a hydroxyalkyl having 1 to 3 carbon atoms; or R³ and R⁴ are coupled together to form an alkylene having 3 to 5 carbon atoms; and
R⁵ is a hydrogen, an alkyl having 1 to 5 carbon atoms, a cycloalkyl having 5 to 7 carbon atoms, a hydroxyalkyl having 1 to 3 carbon atoms, a benzyl, a naphthyl, or a substituted or unsubstituted phenyl.

6. The method according to claim 5, wherein:
R³ is an alkyl having 1 to 5 carbon atoms;
R⁴ is a hydrogen; and
R⁵ is an unsubstituted phenyl, or a phenyl substituted by 1 to 3 substituents, which may be the same or different and selected from the group consisting of an alkyl having 1 to 6 carbon atoms, an alkoxy having 1 to 5 carbon atoms, a hydroxyalkyl having 1 to 3 carbon atoms, an alkoxycarbonyl having 2 to 5 carbon atoms in total, an alkylmercapto having 1 to 3 carbon atoms, an alkylamino having 1 to 4 carbon atoms, a dialkylamino having 2 to 8 carbon atoms in total, a halogen atom, trifluoromethyl, carboxyl, cyano, hydroxyl group, nitro, amino, sulfonyl, and acetamido.

7. The method according to claim 6, wherein said pyrazolone derivative is edaravone or a physiologically acceptable salt thereof or a hydrate thereof.

8. The method according to claim 1, wherein said bisphosphonate active agent and said pyrazolone derivative are administered to said subject simultaneously.

9. The method according to claim 1, wherein said bisphosphonate active agent and said pyrazolone derivative are administered to said subject sequentially.

10. The method according to claim 1, wherein said pulmonary route comprises inhalation.

11. The method according to claim 1, wherein said method is a method of treating said subject for a bone absorption disease.

12. The method according to claim 11, wherein said subject has been diagnosed as suffering from said bone absorption disease.

13. The method according to claim 11, wherein said subject has been diagnosed as being at risk for suffering from said bone absorption disease.

14. The method according to claim 11, wherein said bone absorption disease is osteoporosis, osteopenia, urolithiasis, hypercalcemia, Paget's disease, bone metastasis, multiple myeloma, or neoplastic bone lesion.

15. A pharmaceutical composition comprising a bisphosphonate active agent and a pyrazolone derivative in a physiologically acceptable vehicle.

16. The pharmaceutical composition according to claim 15, wherein said bisphosphonate active agent is a compound of formula (I): 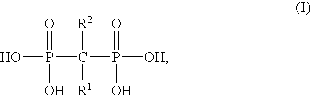
or a physiologically acceptable salt, solvate, hydrate, and prodrug forms thereof, and stereoisomers thereof;
wherein:
R¹ is a hydrogen, —OH, or halogen; and
R² is a halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein each carbon atom of R² may be optionally replaced with a nitrogen or sulfur atom and R² has no more than 3 nitrogen or sulfur atoms in total.

17. The pharmaceutical composition according to claim 16, wherein said bisphosphonate active agent is a compound listed in Table 1.

18. The pharmaceutical composition according to claim 17, wherein said bisphosphonate active agent is alendronate.

19. The according to claim 15, wherein said pyrazolone derivative is a compound of formula (III): 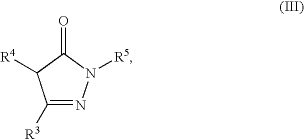
or a physiologically acceptable salt, solvate, hydrate, and prodrug forms thereof, and stereoisomers thereof;
wherein:
R³ is a hydrogen, an aryl, an alkyl having 1 to 5 carbon atoms, or an alkoxycarbonylalkyl having 1 to 6 carbon atoms in total;
R⁴ is a hydrogen, an aryloxy, an arylmercapto, an alkyl having 1 to 5 carbon atoms, or a hydroxyalkyl having 1 to 3 carbon atoms; or R³ and R⁴ are coupled together to form an alkylene having 3 to 5 carbon atoms; and
R⁵ is a hydrogen, an alkyl having 1 to 5 carbon atoms, a cycloalkyl having 5 to 7 carbon atoms, a hydroxyalkyl having 1 to 3 carbon atoms, a benzyl, a naphthyl, or a substituted or unsubstituted phenyl.

20. The pharmaceutical composition according to claim 19, wherein:
R³ is an alkyl having 1 to 5 carbon atoms;
R⁴ is a hydrogen; and
R⁵ is an unsubstituted phenyl, or a phenyl substituted by 1 to 3 substituents, which may be the same or different and selected from the group consisting of an alkyl having 1 to 6 carbon atoms, an alkoxy having 1 to 5 carbon atoms, a hydroxyalkyl having 1 to 3 carbon atoms, an alkoxycarbonyl having 2 to 5 carbon atoms in total, an alkylmercapto having 1 to 3 carbon atoms, an alkylamino having 1 to 4 carbon atoms, a dialkylamino having 2 to 8 carbon atoms in total, a halogen atom, trifluoromethyl, carboxyl, cyano, hydroxyl group, nitro, amino, sulfonyl, and acetamido.

21. The pharmaceutical composition according to claim 20, wherein said pyrazolone derivative is edaravone or a physiologically acceptable salt thereof or a hydrate thereof.

22. The pharmaceutical composition according to claim 15, wherein said pharmaceutical composition is an aerosol.

23. The pharmaceutical composition according to claim 22, wherein said aerosol is an aerosol of liquid particles.

24. The pharmaceutical composition according to claim 22, wherein said aerosol is an aerosol of solid particles.

25. The pharmaceutical composition according to claim 22, wherein said aerosol of solid particles comprises a dry powder.

26. The pharmaceutical composition according to claim 25, wherein said powder comprises particles ranging in size from about 1 μm to about 100 μm.

27. A kit for use in treating a subject suffering from a bone absorption disease condition, said kit comprising:
(a) a bisphosphonate active agent; and
(b) a pyrazolone derivative.

28. The kit according to claim 27, wherein said bisphosphonate active agent is a compound of formula (I): 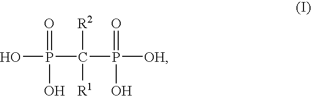
or a physiologically acceptable salt, solvate, hydrate, and prodrug forms thereof, and stereoisomers thereof;
wherein:
R¹ is a hydrogen, —OH, or halogen; and
R² is a halogen, a linear or branched substituted or unsubstituted C₁-C₁₀ alkyl, a linear or branched substituted or unsubstituted C₁-C₁₀ cycloalkyl, a linear or branched substituted or unsubstituted C₁-C₁₀ aryl, a linear or branched substituted or unsubstituted C₁-C₁₀ aralkyl, a substituted or unsubstituted C₁-C₁₀ heterocycloalkyl, or a substituted or unsubstituted C₁-C₁₀ heteroaryl, wherein each carbon atom of R² may be optionally replaced with a nitrogen or sulfur atom and R² has no more than 3 nitrogen or sulfur atoms in total.

29. The kit according to claim 28, wherein said bisphosphonate active agent is a compound listed in Table 1.

30. The kit according to claim 29, wherein said bisphosphonate active agent is alendronate.

31. The kit according to claim 27, wherein said pyrazolone derivative is a compound of formula (III): 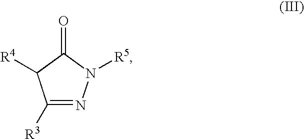
or a physiologically acceptable salt, solvate, hydrate, and prodrug forms thereof, and stereoisomers thereof;
wherein:
R³ is a hydrogen, an aryl, an alkyl having 1 to 5 carbon atoms, or an alkoxycarbonylalkyl having 1 to 6 carbon atoms in total;
R⁴ is a hydrogen, an aryloxy, an arylmercapto, an alkyl having 1 to 5 carbon atoms, or a hydroxyalkyl having 1 to 3 carbon atoms; or R³ and R⁴ are coupled together to form an alkylene having 3 to 5 carbon atoms; and
R⁵ is a hydrogen, an alkyl having 1 to 5 carbon atoms, a cycloalkyl having 5 to 7 carbon atoms, a hydroxyalkyl having 1 to 3 carbon atoms, a benzyl, a naphthyl, or a substituted or unsubstituted phenyl.

32. The kit according to claim 31, wherein:
R³ is an alkyl having 1 to 5 carbon atoms;
R⁴ is a hydrogen; and
R⁵ is an unsubstituted phenyl, or a phenyl substituted by 1 to 3 substituents, which may be the same or different and selected from the group consisting of an alkyl having 1 to 6 carbon atoms, an alkoxy having 1 to 5 carbon atoms, a hydroxyalkyl having 1 to 3 carbon atoms, an alkoxycarbonyl having 2 to 5 carbon atoms in total, an alkylmercapto having 1 to 3 carbon atoms, an alkylamino having 1 to 4 carbon atoms, a dialkylamino having 2 to 8 carbon atoms in total, a halogen atom, trifluoromethyl, carboxyl, cyano, hydroxyl group, nitro, amino, sulfonyl, and acetamido.

33. The kit according to claim 32, wherein said pyrazolone derivative is edaravone or a physiologically acceptable salt thereof or a hydrate thereof.
